(19) United States
(12) Patent Application Publication
(10) Pub. No.: US 2025/0051253 A1
(43) Pub. Date: Feb. 13, 2025

(54) SUPERCRITICAL ETHYLENE EXTRACTION PROCESS FOR SELECTIVELY RECOVERING PHENOLIC COMPOUNDS FROM BIO-CRUDE AND/OR BIO-OIL

(71) Applicant: Research Triangle Institute, Research Triangle Park, NC (US)

(72) Inventors: Michael Carpenter, Research Triangle Park, NC (US); Ofei D. Mante, Research Triangle Park, NC (US); Joseph Michael Weiner, Research Triangle Park, NC (US)

(21) Appl. No.: 18/719,015

(22) PCT Filed: Dec. 16, 2022
(86) PCT No.: PCT/US22/81783

§ 371 Date: Jun. 12, 2024

Related U.S. Application Data
(60) Provisional application No. 63/292,626, filed on Dec. 22, 2021.

(51) Int. Cl. C07C 41/38

(52) U.S. Cl. CPC C07C 41/38

(57) ABSTRACT

A supercritical ethylene extraction process for selectively recovering a phenolic compound from bio-crude and/or bio-oil comprises providing a reactor containing bio-crude and/or bio-oil, wherein the reactor has an operating pressure, introducing an ethylene solvent at the operating pressure to the reactor, and using the ethylene solvent at the operating pressure to extract the extracted portion from the feedstock, wherein the extracted portion comprises the selected phenolic compound at a concentration equal to or greater than 70 wt % on a dry basis.

### CROSS-REFERENCE TO RELATED APPLICATION

This application claims the benefit of U.S. Provisional Patent Application No. 63/292,626, filed on Dec. 22, 2021, the entire content of which is incorporated herein by reference.

### FIELD

The present disclosure describes a process for selectively recovering phenolic compounds from bio-crude using a supercritical extraction solvent. In particular, the supercritical extraction solvent comprises ethylene.

### BACKGROUND

A number of industries including pharmaceuticals, natural essential oils, the polymer industry, and the flavor and fragrance industry have a commercial interest in recovering phenolic fractions from feedstock comprising bio-oil and/or bio-crude. Recovering phenolic fractions is more commercially attractive if high purity phenolic fractions can be recovered efficiently with relatively low environmental impact.

Phenolic fractions have been recovered from bio-oils and biocrude using separation techniques such as liquid-liquid extraction (LLE), distillation, and adsorption (e.g., column chromatography) with varying degree of success. Liquid-liquid extraction techniques using alkaline extraction solvents, such as, for example, dichloromethane, methyl isobutyl ketone, hexane or methyl tertiary butyl either, have been used. However, extraction of valuable phenolic fractions from biomass and biomass derivatives is challenging due to the wide array of similar, co-solvated species. The existence of compound azeotropes complicates extractions, and often traditional solvent methods are unable to achieve desired selectivity.

For example, solvents such as toluene, benzene, acetone, or higher order hydrocarbons may not be able to provide the precise separation necessary to achieve economical operation. Additionally, the need for secondary separation to remove the solvent can result in further losses of valuable product. Therefore, use of these solvents may be costly, not only for the materials themselves, but also for further recycling or disposal costs required to achieve desired product selectivity or to meet environmental emission requirements for organic solvents.

Additionally, recovery processes using supercritical carbon dioxide as an extraction solvent have been reported. Supercritical carbon dioxide extraction uses what is conventionally considered a waste or byproduct of industrial and natural processes to recover phenolic products. Supercritical carbon dioxide extraction generally does not extract sugar, protein, or particulates into phenolic product. However, unfortunately, supercritical carbon dioxide does extract most polar species, including water and organics acids, into the phenolic product. Water and organic acids are undesirable contaminants in extracted phenolic products, which require either subsequent separations or initial feedstock drying procedures, both of which add cost and processing steps thereby reducing operational efficiency.

There is a need for a process that addresses the known challenges with respect to phenolic selectivity, separation efficiency, residual losses, and enhanced purity of the recovered phenolic product as it relates to recovering phenolics from bio-oil/biocrude.

### SUMMARY OF THE DISCLOSURE

In an aspect of the invention, a supercritical ethylene extraction process for selectively recovering a phenolic compound from feedstock comprising bio-crude and/or bio-oil, wherein a concentration of the selected phenolic compound in the extracted portion of the feedstock is greater than 70 wt % on a dry basis, the process comprises: providing a reactor containing feedstock comprising bio-crude and/or bio-oil, wherein the reactor has an operating pressure, introducing an ethylene solvent at the operating pressure to the reactor, and using the ethylene solvent at the operating pressure to extract the extracted portion from the feedstock, wherein the extracted portion comprises the selected phenolic compound at a concentration equal to or greater than 70 wt % on a dry basis.

In another aspect of the invention, a supercritical ethylene extraction process for selectively recovering a phenolic compound from feedstock comprising bio-crude and/or bio-oil, wherein a concentration of water in the extracted portion of the feedstock is less than 10 wt %, the process comprises providing a reactor containing feedstock comprising bio-crude and/or bio-oil, wherein the reactor has an operating pressure, introducing an ethylene solvent at the operating pressure to the reactor, and using the ethylene solvent at the operating pressure to extract the extracted portion from the feedstock, wherein the extracted portion comprises water at a concentration less than 10 wt %.

In a further aspect of the invention, a supercritical ethylene extraction process for selectively recovering a phenolic compound from feedstock comprising bio-crude and/or bio-oil, wherein a moisture content in the extracted portion of the feedstock is about 1.5 to about 5.5 percent moisture, the process comprises providing a reactor containing feedstock comprising bio-crude and/or bio-oil, wherein the reactor has a reactor pressure, introducing an ethylene solvent at a solvent pressure to the reactor, and using the ethylene solvent at the solvent pressure to extract the extracted portion from the feedstock, wherein the moisture content in the extracted portion is about 1.5 to about 5.5 percent moisture.

### BRIEF DESCRIPTION OF THE FIGURES

FIG. 1 is a schematic representation of an exemplary extraction unit showing an exemplary process flow diagram.

FIG. 2 is a set of tables showing the constituent components of extract streams in Example 2 on a dry basis.

FIG. 3 is a chart showing the increased concentration of methoxyphenols in extract from ethylene extraction in comparison to extract from carbon dioxide extraction in Example 3.

FIG. 4 is a chart plotting the concentration of multifunctional phenolics over time on an absolute dry basis in Example 4.

FIG. 5 is a chart plotting the concentration of multifunctional phenolics over time in relative terms in Example 4.

### DETAILED DESCRIPTION

Described herein is an ethylene extraction process for selectively recovering a phenolic compound from feedstock comprising bio-crude and/or bio-oil. The process can enable the concentration of the selected phenolic compound in the extracted portion of the feedstock to be greater than 70 wt % on a dry basis. In the process, a reactor containing feedstock comprising bio-crude and/or bio-oil is provided. The reactor has an operating pressure. An ethylene solvent is introduced to the reactor at the operating pressure of the reactor. The ethylene solvent and the reactor are pressurized to an operating pressure that allows the ethylene to be in a supercritical state.

Supercritical ethylene can be used to selectively extract phenolic compounds (i.e., mono and polyphenols), along with other aromatics, from bio-oil or bio-crude while excluding water, organic acids, protein, sugar, and other macromolecules that are considered contaminants. The highly selectivity process enables separation of target chemicals from wet substrates, like biocrude or wet biomass, thereby avoiding costly and inefficient drying operations. Advantageously, the extraction is predictable and tunable and changes the bulk physical properties of the source material as little as possible.

A supercritical fluid is a substance at a temperature and pressure above its critical point, where distinct liquid and gas phases do not exist but below the pressure needed to compress the fluid into a solid. In embodiments of the ethylene extraction process, the operating pressure can be about 50 to about 300 bar, about 50 to about 200 bar, or about 50 to about 100 bar. Given the foregoing, the skilled artisan will understand that the operating pressure can be any pressure between about 50 and about 300 bar, for example, 50, 70, 90, 100, 110, 130, 150, 170, 190, 200, 210, 230, 250, 270, 290, 300 bar or any pressures therebetween. The operating temperature can be about 9° C. to about 100° C., about 9° C. to about 60° C., or about 9° C. to about 40° C. Given the foregoing, the skilled artisan will understand that the operating temperature can be any temperature between about 9° C. to about 100° C., for example, 9, 10, 20, 30, 40, 50, 60, 70, 80, 90, or 100° C. or any temperatures therebetween. The operating pressure and temperature will affect the density of the ethylene solvent, which may be between about 50-500 kg/m³.

The ethylene solvent removes a portion of the feedstock via solvent extraction. In embodiments, the ethylene solvent can include carbon dioxide or other extraction solvents in the supercritical regime to extract selected phenolic compounds. The removed portion or extracted portion of the feedstock comprises a selected phenolic compound. The compound or compounds can be present at a concentration of 50 wt % to 100 wt %. For example, the compound or compounds can be present at a concentration equal to or greater than 70 wt % on a dry basis. It will be appreciated that in embodiments, the extracted portion can comprise a selected phenolic compound or compounds in concentrations equal or greater than 75 wt % on a dry basis or equal or greater than 80 wt % on a dry basis. The extracted portion can comprise a selected phenolic compound or compounds in concentrations equal or greater than 50 wt %, 55 wt %, 60 wt %, 65 wt %, 70 wt %, 71 wt %, 72 wt %, 73 wt %, 74 wt %, 75 wt %, 76 wt %, 77 wt %, 78 wt %, 79 wt %, 80 wt %, 85 wt %, 90 wt %, 95 wt %, or 100 wt % on a dry basis.

As a person having ordinary skill in the art will understand, the term “dry basis” is an expression of the calculation in which the presence of water (and/or other solvents) is neglected for the purposes of the calculation. Water (and/or other solvents) can be neglected because addition and removal of water (and/or other solvents) are common processing steps, and also happen naturally through evaporation and condensation. Thus, it can be useful to express compositions on a dry basis to remove these effects. In the examples provided herein, the analysis was performed by gas chromatography mass spectrometry (GCMS) on a dry basis.

The extracted portion of the feedstock comprises a selected phenolic compound or compounds that can be recovered for later use and/or application. The phenolic compounds can have a phenolic group, a methoxy group, and substituents such monofunctional methoxyphenols (MPs). The phenolic compound may be a guaiacol or a eugenol. The recovered MP can be used for applications in industries such as flavor and fragrance, pharmaceuticals as methyl, ethyl, propyl, and allyl. These phenolic compounds are referred to as, natural essential oils, and polymers.

Other phenolic compounds with acidic, ketone, aldehyde, and additional hydroxy functionalities can be recovered if desired. While recovery of MPs is described herein, one of ordinary skill in the art will understand that other phenolic compounds can be recovered using the supercritical ethylene extraction process described herein.

The feedstock comprises bio-oil and/or bio-crude. As used herein, the terms “bio-oil” and “bio-crude” can be used interchangeably and are intended to mean the fraction of reaction products obtained from a biomass pyrolysis reaction that is liquid at ambient condition. The liquid-phase products may comprise hydrophilic phase compounds, hydrophobic phase compounds, or a mixture of hydrophilic and hydrophobic phase compounds. The biomass starting material used for pyrolysis can include a wide variety of biological resources. For example, the term biomass can take on the meaning set forth in the Energy Policy Act of 2005. Thus, the term “biomass” can mean: any lignin waste material that is segregated from other waste materials and is determined to be nonhazardous by the Administrator of the Environmental Protection Agency and any solid, nonhazardous, cellulosic material that is derived from—(A) any of the following forest-related resources: mill residues, precommercial thinners, slash, and brush, or nonmerchantable material; (B) solid wood waste materials, including waste pallets, crates, dunnage, manufacturing and construction wood wastes (other than pressure-treated, chemically-treated, or painted wood wastes), and landscape or right-of-way tree trimmings, but not including municipal solid waste (garbage), gas derived from the biodegradation of solid waste, or paper that is commonly recycled; (C) agriculture wastes, including orchard tree crops, vineyard, grain, legumes, sugar, and other crop by-products or residues, and livestock waste nutrients; or (D) a plant that is grown exclusively as a fuel for the production of electricity. Exemplary plants useful as a fuel for energy production include switchgrass, miscanthus, energy canes, sorghum, willows, poplar, and eucalyptus. For example, the biomass starting material for the pyrolysis process may comprise a lignocellulosic material. Exemplary pyrolysis processes are described in commonly owned U.S. Pat. No. 9,944,857, U.S. Patent Application Publication No. 2015/0307786, and U.S. Patent Application Publication No. 2016/0222298, the entire contents of which are incorporated by reference herein.

In addition to high selectivity for intended phenolic compounds, advantageously, in embodiments, the ethylene extraction process described herein largely avoids extraction of water and sugars. For example, in embodiments, the supercritical ethylene extraction process results in a concentration of water in the extracted portion of the feedstock of less than 10 wt %. For example, the extraction process may result in an extracted portion comprising water at a concentration less than 7 wt %, less than 5 wt % or less than 2 wt %.

This ethylene extraction process can selectively isolate methoxyphenols from biocrude or supercritical carbon dioxide extract, respectively. The process can operate in either a semi-batch or continuous set up. In the process, ethylene solvent is bubbled through the feedstock at temperatures and pressures above the critical point. A pressure relief mechanism can be used to condense the extracted portion of the feedstock. In embodiments, the process has shown over 70% selectivity for methoxyphenols in biocrude. Additionally, in embodiment, the extraction process can provide relatively complete water and sugar rejection.

Often supercritical solvents are permanent gasses, meaning that separation and later recycling of the solvent after extraction can be achieved through pressure reduction whereby the solvent expands to a gas and the extracted portion remains as a liquid. Advantageously, there is little to no retention of the solvent in the extract to contaminate the product. Avoiding additional separation processing steps is efficient and cost effective. Additionally, a purer product is generally more attractive to consumers.

Supercritical CO₂ extraction is a known process in which CO₂ is used as an extraction solvent in its supercritical state. Using supercritical ethylene as an extraction solvent rather than CO₂ can provide the advantages of using supercritical CO₂ while also avoiding some of the downsides or challenges of supercritical CO₂ extraction. For example, the supercritical CO₂ extraction process extracts most polar species, including water and organics acids. Water and organic acids require either subsequent separations or initial feedstock drying procedures, both of which add operational complexity and cost. In contrast, supercritical ethylene extraction does not extract water or organic acids. Thus, replacing supercritical CO₂ extraction with supercritical ethylene extraction in a manufacturing or processing setting can enable more efficient and cost-effective extraction and potentially enable elimination of a processing unit, such as, for example, a dryer.

Using ethylene as a supercritical solvent for extraction provides many of the benefits of carbon dioxide supercritical extraction while limiting the drawbacks. For example, when supercritical ethylene is used for extraction with feedstock comprising biocrude, water and organic acids are extracted in very small amounts. At the same time, supercritical ethylene maintains extraction selectivity towards targeted phenolic compounds.

In fact, supercritical carbon dioxide extraction removes enough water from the feedstock that the extracted portion separates into two phases. Extraction of water at such high concentrations negatively affects the product stream composition by diluting the constituents and by hindering further separation using phase-separating techniques. The desired products do not separate cleanly into one phase or the other, requiring separation of both phases to recover all product (or as much product as possible). The presence of water in the extracted portion also negatively affects the viscosity of the substrate, making it harder to extract subsequent material.

### EXAMPLES

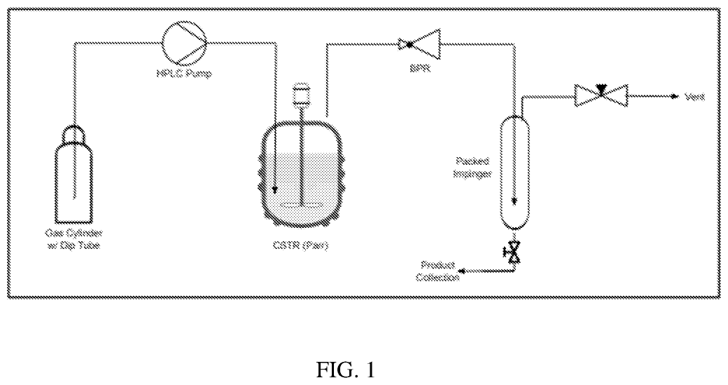

System overview: The exemplary bench scale supercritical extraction unit was used for the removal of methoxy phenols from biocrude. FIG. 1 is a schematic representation of the extraction unit showing a process flow diagram. The unit was fed by a cylinder of solvent liquid containing a dip tube. The solvent liquid could be solvents such as, for example, CO₂ or ethylene. The outlet of the gas cylinder was regulated isobaric to the tank pressure to maintain the solvent fluid in the liquid phase. The fluid was plumbed to an HPLC pump (Gilson 305) which delivered the fluid to the stirred tank at a rate up to 50 mL/min. The extraction vessel was a 500 mL parr reactor with an impellor that continuously agitated the extractable material. The solvent fluid entered the vessel via a dip tube such that it was bubbling through the biocrude. The biocrude was obtained from a catalytic fast pyrolysis of woody biomass. The solvent fluid and solvated extract exited the top of the vessel and were carried through a back pressure regulator which dropped the pressure from supercritical conditions to around 100 psi. The back pressure regulator was set to maintain the solvent fluid in its supercritical state. The exiting solvent fluid flashed to vapor upon the drop of pressure causing the extracted material to drop out as a liquid. The extract liquid was collected in the impinger which was packed to knock out any aerosols. The vapor then exited through a needle valve, which maintained the impinger pressure, to vent.

Exemplary experimental procedure: The following is a general description that was used for the examples shown herein. Experiments were begun with an empty and clean extraction tank and collection impinger. Nominally 200 g of biocrude was loaded into the tank. The vessel was sealed and tightened, and all tubing was secured. The back pressure regulator was set to the desired pressure of extraction. The pressure was 100 bar for both CO₂ and ethylene for exemplary experiments. The vessel was brought to the gas cylinder tank pressure and the temperature controller was started to bring the vessel to thermal operating conditions, 40° C. Once the unit reached the temperature set point, the HPLC pump was started at the operating flow rate. The pressure gradually increased until it reached the back pressure regulator set point. At this point, the fluid inside the extraction tank was in a supercritical state. The flow out of the vessel equilibrated with the HPLC pump and product collection began. The needle valve at the outlet of the collection impinger was adjusted to maintain nominally 100 psi inside. The process was continued until the product was fully extracted or the desired time on stream was reached. Samples were taken from the product collection vessel at regular intervals through a sample port at the bottom. At the end of the experiment, the unit was slowly depressurized, the product vessel cleaned to collect residual extract, and the extracted biocrude was sampled and weighed.

All the samples, including the biocrude pre and post extraction, were analyzed via GCMS analysis for qualitative and quantitative analysis. Karl Fischer titration was performed to determine the water content. The mass closure was calculated, and the extraction efficiency was determined from the GC-MS analysis for each compound of interest.

### Example 1

An exemplary experiment was run to determine and measure the amount of water present in extract from supercritical ethylene extraction in comparison to extract from supercritical carbon dioxide extraction. Table 1 shows moisture analysis results of extract from ethylene extraction and extract from carbon dioxide extraction—both the top and bottom phases. As can be seen, extract from ethylene supercritical extraction had only 4.7% of the water that extract from carbon dioxide extraction did at the same flowrate.

TABLE 1

| Representative Sample | Percent Water | Mass Water (g) |
|---|---|---|
| 30 mL/min ethylene | 1.5 | 0.06 |
| 30 mL/min CO₂ bottom | 9.4 | 0.15 |
| phase | | |
| 30 mL/min CO₂ top phase | 68.9 | 1.18 |

The carbon dioxide extract includes enough water that it separates into two phases. The separation negatively affects the product stream composition not only by diluting the constituents, but also by hindering any further separation by phase-separating components. The desired products do not separate cleanly into one phase or the other, requiring separation of both phases to recover all product. Extraction of water also negatively affects the viscosity of the substrate, making it harder to extract subsequent material.

### Example 2

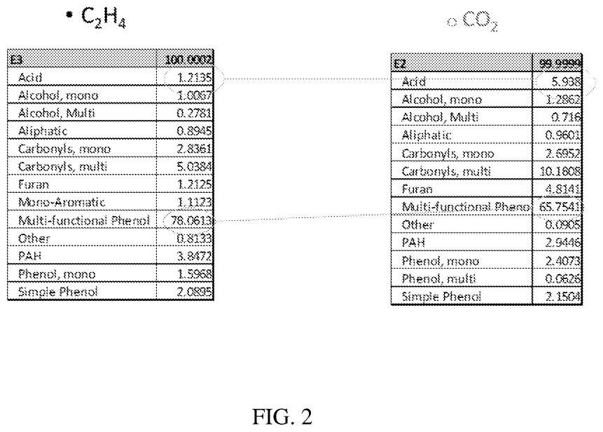

An exemplary experiment was run to determine and measure the composition of the extract from supercritical ethylene extraction in comparison to extract from supercritical carbon dioxide extraction on a dry basis. FIG. 2 is a set of tables showing the constituent components on a dry basis. As can be seen in FIG. 2, the extract from ethylene extraction has reduced organic acids relative to extract from carbon dioxide extraction and increased poly and simple phenols relative to extract from carbon dioxide extraction. While this results in a lower overall product volume, the percentage of desired methoxyphenols, which is modelled as “Multifunctional Phenol” is significantly higher. The analysis is done by GCMS on a dry basis, which artificially concentrates the CO₂ stream.

### Example 3

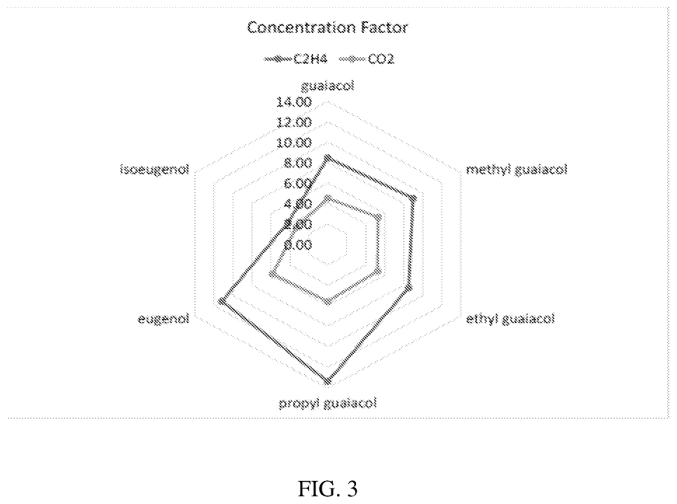

An exemplary experiment was run to determine the effect of the presence of water in the extract stream. FIG. 3 is a chart showing the increased concentration of methoxyphenols in extract from ethylene extraction in comparison to extract from carbon dioxide extraction. The data in FIG. 2 is provided on a dry basis, thus the effect of water is not apparent in the data of FIG. 2. The data in FIG. 3 is not provided on a dry basis, thus the effect of water dilution on the extract streams is shown. The water dilution results in concentrations of phenolic compounds that are lower than GCMS analysis on a dry basis would otherwise lead one to believe.

The rejection of water by supercritical ethylene solvent provides opportunities for extracting wet biomass/bioproducts, which lowers the time and energy required to produce extracts. Moreover, subsequent drying or drying prior to extraction can have a pronounced detrimental effect on the yield, such as in turmeric, where the curcumin content can go down by 90% upon drying. Accordingly, supercritical ethylene extraction can provide an advantage over state-of-the-art practices.

### Example 4

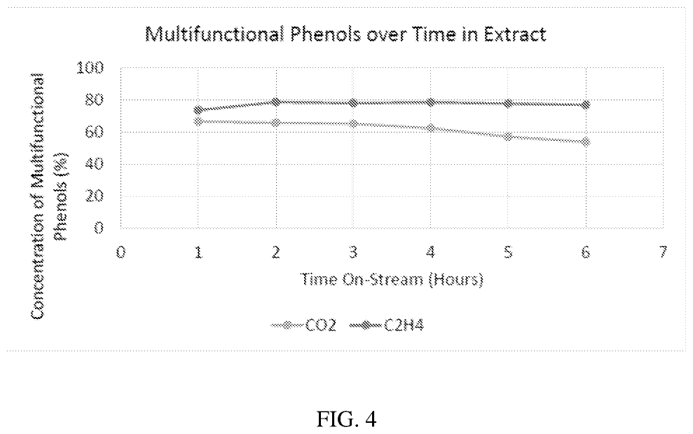

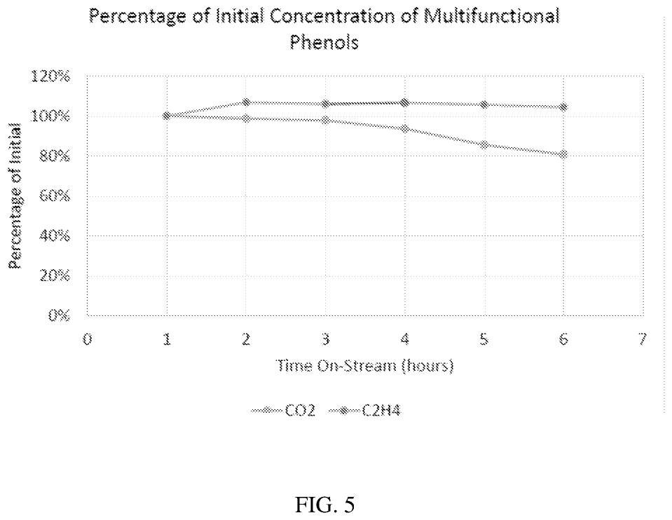

An exemplary experiment was run to determine the effect of time on the percentage of multifunctional phenolics in the product stream. FIG. 4 is a chart plotting the concentration of multifunctional phenolics over time on an absolute dry basis. FIG. 5 is a chart plotting the concentration of multifunctional phenolics over time in relative terms. As can be seen, there is a drop off in multifunctional phenolics in the CO₂ extract stream over time. However, the concentration of multifunctional phenolics in the ethylene extract stream stays relatively constant over time. For example, once the concentration of multifunctional phenolics in the ethylene extract stream reached the peak concentration of about 78%, the concentration varied by 5% or less over the operating time of 6 hours. A person having ordinary skill in the art will understand that this experiment is an example of supercritical extraction with ethylene. Operating times can vary and may include times ranging from, for example, 4 hours to 24 hours. Additionally, operation may be continuous or batch operation.

While not being bound by theory, the proposed hypothesis for this phenomenon is that because ethylene leaves water, acids, and other low viscosity liquids in the bulk material, there is relatively little change in viscosity of the bulk and of the solvent. The lack of viscosity change allows for greater relative penetration of the solvent over time and a more complete extraction.

Numerous modifications and variations of the present disclosure are possible in view of the above teachings. It is understood that within the scope of the appended claims, the disclosure may be practiced otherwise than as specifically described herein.

It should be understood that the above description is only representative of illustrative embodiments and examples. For the convenience of the reader, the above description has focused on a limited number of representative examples of all possible embodiments, examples that teach the principles of the disclosure. The description has not attempted to exhaustively enumerate all possible variations or even combinations of those variations described. That alternate embodiments may not have been presented for a specific portion of the disclosure, or that further undescribed alternate embodiments may be available for a portion, is not to be considered a disclaimer of those alternate embodiments. One of ordinary skill will appreciate that many of those undescribed embodiments, involve differences in technology and materials rather than differences in the application of the principles of the disclosure. Accordingly, the disclosure is not intended to be limited to less than the scope set forth in the following claims and equivalents.

1. A supercritical ethylene extraction process for selectively recovering a phenolic compound from feedstock comprising bio-crude and/or bio-oil, wherein a concentration of the selected phenolic compound in the extracted portion of the feedstock is greater than 70 wt % on a dry basis, the process comprising:
providing a reactor containing feedstock comprising bio-crude and/or bio-oil, wherein the reactor has an operating pressure,
introducing an ethylene solvent at the operating pressure to the reactor, and
using the ethylene solvent at the operating pressure to extract the extracted portion from the feedstock, wherein the extracted portion comprises the selected phenolic compound at a concentration equal to or greater than 70 wt % on a dry basis.

2. The extraction process of claim 1, wherein the extracted portion comprises the selected phenolic compound at a concentration equal to or greater than 75 wt % on a dry basis.

3. The extraction process of claim 1, wherein the extracted portion comprises the selected phenolic compound at a concentration of about 70 wt % to about 90 wt % on a dry basis.

4. The extraction process of claim 1, wherein the supercritical ethylene extraction process operates with the same ethylene solvent for an amount of time and the extracted portion comprises the selected phenolic compound at a concentration equal to or greater than 70 wt % on a dry basis for a time of up to 4 hours.

5. The extraction process of claim 4, wherein the extracted portion comprises the selected phenolic compound at a concentration equal to or greater than 70 wt % on a dry basis for a time of up to 6 hours.

6. The extraction process of claim 1, wherein the selected phenolic compound comprises methoxyphenols.

7. The extraction process of claim 1, wherein the supercritical ethylene extraction process operates with the same ethylene solvent for an amount of time and the viscosity of the ethylene solvent varies by less than or equal to 5% for a time of up to 4 hours.

8. (canceled)

9. The extraction process of claim 1, wherein the ethylene solvent has a density of about 50-500 kg/m³ at the operating pressure.

10. A supercritical ethylene extraction process for selectively recovering a phenolic compound from feedstock comprising bio-crude and/or bio-oil, wherein a concentration of water in the extracted portion of the feedstock is less than 10 wt %, the process comprising:
providing a reactor containing feedstock comprising bio-crude and/or bio-oil, wherein the reactor has an operating pressure,
introducing an ethylene solvent at the operating pressure to the reactor, and
using the ethylene solvent at the operating pressure to extract the extracted portion from the feedstock, wherein the extracted portion comprises water at a concentration less than 10 wt %.

11. The extraction process of claim 10, wherein the extracted portion comprises water at a concentration less than 7 wt %.

12. The extraction process of claim 11, wherein the extracted portion comprises water at a concentration less than 5 wt %.

13. The extraction process of claim 11, wherein the extracted portion comprises water at a concentration less than 2 wt %.

14. The extraction process of claim 10, wherein the extracted portion comprises water at a concentration of about 0 wt % to about 10 wt %, or about 1 wt % to about 7 wt %, or about 1 wt % to about 5 wt %.

15. A supercritical ethylene extraction process for selectively recovering a phenolic compound from feedstock comprising bio-crude and/or bio-oil, wherein a moisture content in the extracted portion of the feedstock is about 1.5 to about 5.5 percent moisture, the process comprising:
providing a reactor containing feedstock comprising bio-crude and/or bio-oil, wherein the reactor has a reactor pressure,
introducing an ethylene solvent at a solvent pressure to the reactor, and
using the ethylene solvent at the solvent pressure to extract the extracted portion from the feedstock, wherein the moisture content in the extracted portion is about 1.5 to about 5.5 percent moisture.

16. The ethylene extraction process of claim 1, wherein the operating pressure is about 50 bar to about 300 bar.

17. The ethylene extraction process of claim 16, wherein the operating pressure is about 50 bar to about 200 bar.

18. The ethylene extraction process of claim 17, wherein the operating pressure is about 50 bar to about 100 bar.

19. The ethylene extraction process of claim 1, wherein the reactor has an operating temperature, and the operating temperature is about 9° C. to about 100° C.

20. The ethylene extraction process of claim 19, wherein the operating temperature is about 9° C. to about 60° C.

21. The ethylene extraction process of claim 19, wherein the operating temperature is about 9° C. to about 40° C.
